(19) United States
(12) Patent Application Publication
(10) Pub. No.: US 2021/0102914 A1
(43) Pub. Date: Apr. 8, 2021

(54) MEMBRANE-BASED WATER QUALITY SENSOR

(71) Applicant: Hach Company, Loveland, CO (US)

(72) Inventors: Corey Salzer, Fort Collins, CO (US); Vishnu Rajasekharan, Fort Collins, CO (US); Dan Kroll, Fort Collins, CO (US); John Lee, Fort Collins, CO (US)

(21) Appl. No.: 17/127,296

(22) Filed: Dec. 18, 2020

Related U.S. Application Data
(63) Continuation of application No. 15/766,806, filed on Apr. 6, 2018.
(60) Provisional application No. 62/239,384, filed on Oct. 9, 2015.

(51) Int. Cl. G01N 27/404; G01N 27/49; G01N 33/18; G01N 27/407

(52) U.S. Cl. CPC G01N 27/4045; G01N 27/4072; G01N 33/182; G01N 27/49

(57) ABSTRACT

An embodiment provides an amperometric sensor, including: a housing containing an inner fill solution; an electrode bathed in the inner fill solution; and a membrane in intimate contact with the electrode; the electrode being formed as a non-compact or porous structure on the membrane; whereby a spatial relationship of the electrode and the membrane is constant. Other embodiments are described and claimed.

### CROSS-REFERENCE TO RELATED APPLICATION

This application is a continuation application of co-pending U.S. patent application Ser. No. 15/766,806, entitled “MEMBRANE-BASED WATER QUALITY SENSOR,” filed Apr. 6, 2018, which is a National Phase application of WO 2017/062849, entitled “MEMBRANE-BASED WATER QUALITY SENSOR,” filed Oct. 7, 2016, which claims priority to provisional U.S. Patent Application Ser. No. 62/239,384, entitled “MEMBRANE-BASED WATER QUALITY SENSOR,” filed Oct. 9, 2015, the contents of all of which are hereby incorporated by reference as if set forth in their entirety.

### FIELD

The subject matter described herein is in the general field of water quality measurement, specifically for monitoring chlorine and/or monochloramine levels by using new electrochemical sensor designs and methods.

### BACKGROUND

Membrane-based amperometric sensors or probes (the terms sensor and probe are used interchangeably herein) for chlorine detection in water employ an electrode in close proximity to a gas diffusion membrane layer with an interposed region comprised of electrolyte, buffer, and/or redox mediator. Conventional designs typically consist of a flexible polymer membrane stretched across a fixed electrode. The membrane provides protection and analyte selectivity to the device and acts as a barrier to retain inner solution electrolyte, buffer and/or redox mediator(s). Electrochemical reactions occur at the electrode, which gives rise to a current response proportional to the analyte concentration.

The process of analyte transport/diffusion through the membrane, across the solution region, and to the electrode surface is mass transport dependent. Changes in the mass transport characteristics of any or all of these components impact the amperometric measurement of the analyte (i.e., current measurement). Changes in pressure and sample flow, physical impact or deformation of the membrane can cause a change in the spatial arrangement of the membrane-electrolyte-electrode dimensions, which can cause an erroneous change in the measurement of the analyte. This is a common issue for conventional membrane-based sensors and has been an impediment to expanding the application space, e.g., for in-pipe applications or other placements that experience pressure variations. Because of conventional sensor susceptibility to pressure fluctuations, sensors of this type are often utilized under isobaric conditions (i.e., atmospheric pressure). Osmotic pressure can also result in a change in the spatial arrangement of membrane-to-electrode, thereby changing the mass transport characteristic under which the sensor was calibrated, resulting in erroneous measurement of the analyte.

Additionally, the calibration stability and operational life of membrane-based amperometric probes can depend on the amount of electrolyte/reagent present in the interposed region between the electrode and membrane. Degradation, depletion, and/or loss of the inner fill solution may result in the need for frequent calibration and maintenance of the sensor.

### BRIEF SUMMARY

In summary, one embodiment provides an amperometric sensor, comprising: a housing containing an inner fill solution; an electrode bathed in the inner fill solution; and a membrane in intimate contact with the electrode; the electrode being formed as a non-compact or porous structure on the membrane.

Another embodiment provides an amperometric sensor, comprising: a housing containing an inner fill solution; an electrode bathed in the inner fill solution; an electrode contact disposed within the housing and coupled to the electrode; probe electronics coupled to the electrode contact; and a membrane in intimate contact with the electrode; the electrode being formed as a non-compact or porous structure on the membrane.

A further embodiment provides a method, comprising: forming an amperometric sensor having a non-compact or porous measuring electrode in intimate contact with a gas diffusion membrane; said non-compact or porous measuring electrode integrated with the gas diffusion membrane by a technique selected from the group consisting of printing, depositing, or adhering the non-compact or porous membrane with or to the gas diffusion membrane such that the two components move in unison and as a unified element in operation.

A further embodiment provides an amperometric sensor, comprising: a housing containing an inner fill solution; an electrode bathed in the inner fill solution; and a membrane separating the inner fill solution from an exterior environment; the electrode being formed as a non-compact or porous structure; whereby a spatial relationship of the electrode and the membrane is substantially constant.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

FIG. 1 illustrates an example sensor having a membrane intimately connected onto an electrode which is not solid according to an embodiment.

FIG. 2(A-I) illustrates example electrodes of a sensor according to an embodiment.

FIG. 3(A-B) illustrates an example sensor according to an embodiment.

FIG. 4 illustrates an example sensor according to an embodiment.

FIG. 5(A-C) illustrates an example sensor having a support structure according to an embodiment.

FIG. 6(A-B) illustrates an example sensor having a guard electrode according to an embodiment.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of example embodiments. One skilled in the relevant art will recognize, however, that various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

The embodiments described herein alleviate the above-described shortcomings of conventional membrane-based sensors and provide a membrane-based sensor with reduced sensitivity to changes in sample pressure and other forces that cause changes in the membrane-electrolyte-electrode spatial arrangement in conventional sensors. The various embodiments also decrease the need to maintain the probe, conventionally required due to electrolyte/reagent limitations in the current state of the art.

In an embodiment one or more electrode(s) for a membrane-based electrochemical sensor are constructed onto a gas diffusion membrane for use in determination of chlorine in water. A conductive electrode material can be printed, deposited, integrated, adhered to, or otherwise placed in intimate contact with a gas diffusion membrane such that the two components move in unison and as a unified element with enhanced robustness. Vapor deposition is a suitable mechanism of forming the electrode(s) on the membrane, for example.

Referring to FIG. 1, illustrated is cross-sectional view of a sensor 100 having a membrane 101 intimately connected onto an electrode 102 which is not solid. The electrode 102 includes at one end an electrode contact 105, which in turn runs lengthwise up the sensor housing 104 and provides a signal (e.g., current) to probe electronics (not illustrated) for determining water quality (e.g., chlorine measurement). The electrode 102 is intimately connected at another end to the membrane 101 and the electrode 102 contains gaps such as holes or pores or circuitous openings allowing paths of transit between the inner fill solution 103 and the membrane 101.

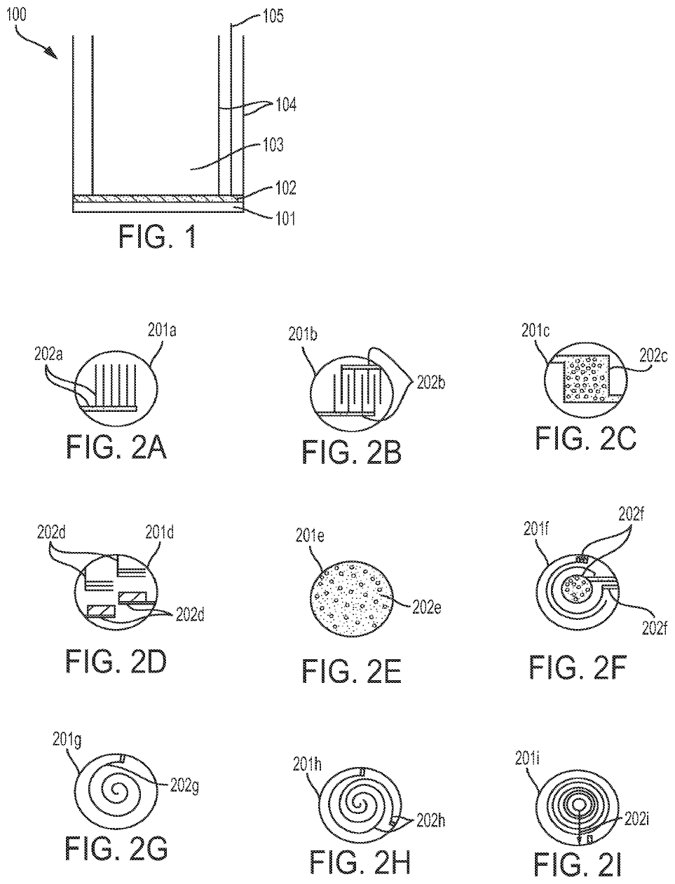

FIG. 2(A-I) illustrate various forms of electrode/membrane configurations suitable for achieving the described effects of the various embodiments. As illustrated in FIG. 2(A-I), various patterns of electrode structures are shown in an end view, where the electrode is in intimate connection or contact with a membrane forming an electrode-membrane structure.

FIG. 2A illustrates a pattern electrode 202a in intimate contact with a membrane 201a. FIG. 2B illustrates patterned electrodes 202b that are interconnected with one another and in intimate contact with a membrane 201b.

FIG. 2C illustrates a non-compact or porous electrode 202c that is in intimate contact with a membrane 201c. The non-compact or porous electrode 202c covers less than the entire membrane 201c.

FIG. 2D illustrates a pattern of four electrodes (collectively indicated at 202d) in intimate contact with a membrane 201d. As illustrated, the electrodes 202d may be patterned or porous. FIG. 2D illustrates use of two patterned and two porous electrodes 202d.

FIG. 2E illustrates a non-compact or porous electrode 202e that is in intimate contact with a membrane 201e. The non-compact or porous electrode 202e covers substantially the entire membrane 201d as viewed from the end of the sensor.

FIG. 2F illustrates a combination of trace electrodes (formed in concentric pattern) around a centrally located porous electrode, collectively indicated at 202f Here, the membrane 201f is in intimate contact with the electrodes 202f.

FIG. 2G illustrates another embodiment in which a pattern or trace electrode 201g, here shown as a spiral structure, is formed on the membrane 201g.

FIG. 2H likewise illustrates a pattern or trace arrangement, wherein the sensor of FIG. 2H includes two spiral or trace pattern electrodes (collectively indicated at 202h) that are in intimate contact with the membrane 201h of the sensor.

FIG. 2I illustrates a patterned electrode 202i in which circles of increasing diameter are intimately contacted to a membrane 201i, where the circles of the electrode 202i share a contact point, as illustrated.

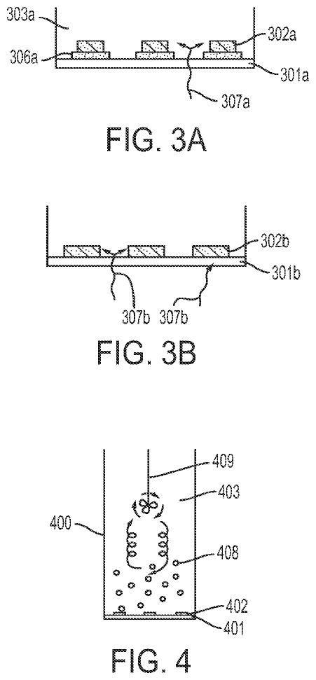

FIG. 3A illustrates a side view diagram or cross section of an electrode-membrane structure integrated into a probe. The membrane 301a is connected to an insulating layer 306a having electrode structures 302a on the side opposite the membrane 301a. As illustrated, an analyte 307a, e.g., chlorine, moves across the membrane 301a and into the inner fill solution 303a to interact with the electrode 302a.

FIG. 3B illustrates a similar structure with a non-compact electrode 302b connected intimately with the membrane 301b itself. Again, an analyte 307b is free to cross the membrane 301b.

FIG. 4 illustrates an embodiment of a probe 400 includes an internal stirring or agitating mechanism 409 to force convection to inner fill solution 403. Particulates 408 such as cleaning beads may be included to facilitate cleaning of the inner electrode surfaces of electrodes 402 in intimate contact with the membrane 401.

In an embodiment, inner fill solution 403 may be stirred or otherwise agitated by agitating mechanism 409 to maintain homogenous solution and replenish inner fill solution 403 to electrode(s) 402. This can prolong the useful performance characteristics of the sensor by maintaining a uniform inner fill solution 403 to the electrode(s) 402. Internal buffer structures and/or the use of micro- and/or nanoelectrode structures will assist with minimizing influence of inner flow rate. Regeneration of the inner fill solution 403 components through electrochemical/chemical methods will also enable to enhance the longevity of the sensor.

In another embodiment, the stirred inner fill solution 403 may contain beads or particulates 408 which can move with the agitated inner fill solution 403 and contact the surface of the electrode(s) 402. These beads or particulates 408 assist in maintaining a clean surface on the electrode(s) 402 to avoid measurement errors cause by fouling or contamination of the electrode(s) 402.

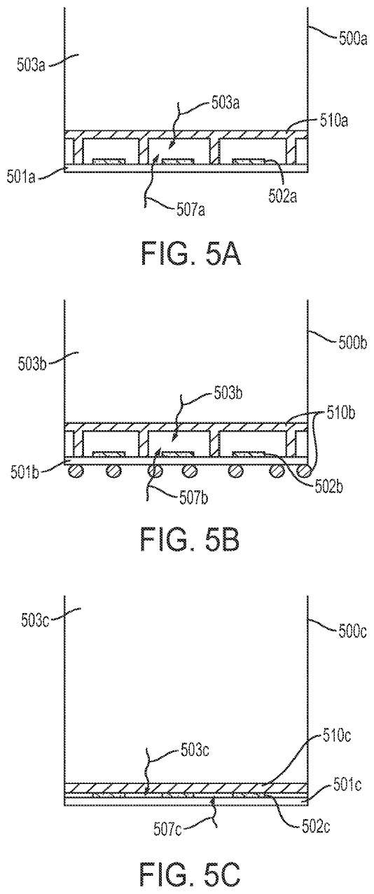

As shown in FIG. 5A, an embodiment includes a physical support structure 510a to support the electrode-membrane structure of the probe 500a. As illustrated in FIG. 5A, the support structure 510a is non-compact or permits the inner fill solution 503a to freely move between an upper chamber and a lower part, i.e., nearest to the electrode 502a. Again, a membrane 501a permits an analyte 507a to enter the probe and interact with the electrode 502a.

In an embodiment, the membrane-electrode can be supported along its membrane side by physical support structure 510a, which may be a compact or non-compact support structure. A support structure 510a is employed in an embodiment to reduce or eliminate movement of the membrane-electrode structure with the intent of prolonging the operational integrity of the membrane-electrode structure. A like arrangement to support the membrane can be employed on the electrode side of the membrane so as to improve the integrity of the membrane-electrode material during changes in sample pressure and flow as well as to protect from physical damage (abrasion, impacts, etc.). Such internal support (refer to FIG. 5B, described further herein) can be an insulating non-compact physical structure residing across the entirety of the active membrane-electrolyte structure. The internal and external support structure(s) may also be constructed such that they are interlaced with the patterns of the membrane-electrode(s). This facilitates support without interruption of critical mass transport and electrochemical processes.

In FIG. 5B, it can be appreciated that the probe 500b may include non-compact support structure(s) 510b that extends or is provided beneath the membrane 501b. The support structure 510b permits movement of the analyte 507b through the membrane 501b and interaction with the inner fill solution 503b and electrode 502b.

The support structure 510c may be insulated, as illustrated in FIG. 5C. The probe 500c illustrated at 5C includes an electrode 502c that is placed between a membrane 501c and the support structure 510c. Analyte 507c moves across membrane 501c and into inner fill solution 503c to interact with electrode 502c.

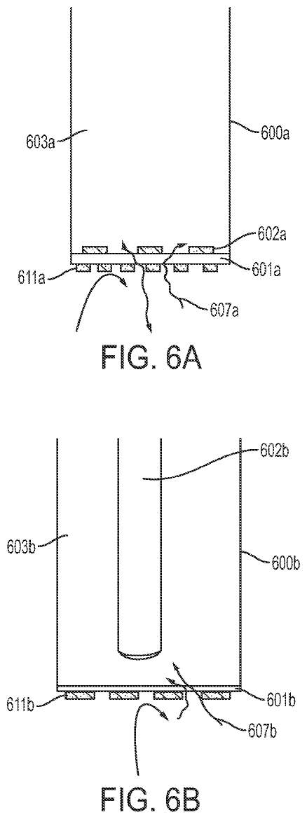

FIG. 6(A-B) illustrates side views in cross section of a probe that includes guard electrodes intimately connected to the membrane on the sample side of a membrane for deterring interfering species from crossing the membrane.

In an embodiment, interfering redox species can be blocked from passing the gas diffusion membrane by the incorporation of a guard electrode on the outer region of the membrane. A porous conductive electrode (e.g., grid, mesh, foam, array, porous plate, etc.) can be positioned on the outside (sample side) or formed on the outer portion of the gas diffusion membrane. This guard electrode is polarized at a potential which induces electrochemical reaction of the interfering species at/near the membrane, preventing the interfering species from passing through the membrane and into the electrolyte/electrode region. The products of the redox reaction of the interfering species may pass the membrane, but will no longer interfere with the measurement of the analyte. The potential of the guard electrode does not affect the analyte of interest, which can pass through the membrane and into the sensing region of the electrode. The non-compact guard electrode may also provide physical support for the membrane.

The membrane-electrode composition can be tuned to vary the mass transport characteristics of the analyte and interferant. The mass transport behavior of ionic versus neutral characteristic of the analyte species of interest will be different depending on the type of the membrane-electrode support. For example, at pH 9 the chloramine species is in its neutral form NH₂Cl whereas the free chlorine is predominantly in its ionic form OCl⁻. This difference in the analyte characteristic will render in varying mass transport rates that can be registered as unique signatures for these analytes.

As shown in FIG. 6A, the probe 600a includes a measuring electrode 602a in intimate contact with a membrane 601a. As with other embodiments, an analyte 607a moves across the membrane 601a interacts with inner fill solution 603a. However, the guard electrodes 611a act to interfere selectively with interfering species, e.g., via redox reaction, preventing their transit across the membrane 601a and entry to the inner fills solution 603a.

FIG. 6B illustrates a probe 600b in which the electrode 602b is positioned within the inner fill solution 603b but is not in intimate contact with the membrane 601b. Here, guard electrodes 611b may again be positioned on an outer surface of the membrane 601b to selectively permit analyte 607b entry into the inner fill solution 603b, whereas interfering species are prevented entry.

In FIG. 6B, the electrode 602b is in a constant (distance) relationship with the membrane 601b because the membrane does not move relative to the electrode 602b. The electrode 602b may be formed as a non-compact or porous structure, as described herein.

As illustrated, e.g., in FIG. 2(A-I), the electrode(s) can be positioned across the whole of the membrane or can be patterned as macro-, micro-, or nano-patterned structures. Single, multiples, continuous, or interlaced structures may comprise the electrode(s), as shown by way of example in FIG. 2(A-I).

The conductive electrode(s) on the membrane are non-compact in form (e.g., metallic sponge). A non-compact form allows passage of analyte to the active region near the electrode(s) and in contact with the inner fill solution where sample conditioning occurs. The conductive electrode(s) may be in direct contact with the membrane or there may be other material(s) between the electrode(s) and the membrane, such as insulating layer or adhesion-assisting layer(s) (e.g., as illustrated in FIG. 3A). An insulating layer acts as a spacer that provides the distance and time for the analyte to mix with the reagents like iodide, if any, in the inner fill solution prior to approach of electrode(s) electrochemically active region. The membrane-electrode structure can be assembled in an electrochemical device such that the membrane is in contact with the aqueous sample and the electrode(s) are on the inner side of the sensor, as for example illustrated in FIG. 1. The electrode(s) may be in contact with electrolyte, reagents, mediators, buffers, etc.

In any embodiment, the region containing the inner fill solution may be microliters in volume or liters in volume. A large reservoir of inner fill solution can extend the life of the sensor and reduce time between necessary maintenance. In contrast, the inner fill solution in the electrode region in conventional sensors is quite small. A larger volume of inner fill solution may allow for reduced concentration of electrolyte/buffers/reagents in inner fill solution and thereby reduce some of the osmotic pressure which can occur in conventional type sensors and gives rise to measurement errors and/or sensor damage.

Embodiments described herein provide arrangements whereby the performance errors and changes caused by conditions which impact the electrode-membrane arrangement in a chlorine sensor are reduced or minimized by making the electrode an integral part of the membrane. The relationship of the electrode(s) to a membrane is a constant by the described method. Issues, such as pressure fluctuations, which can change the relationship of the membrane to the electrode, are mitigated by the described invention. Impact due to changes in the membrane-to-electrode arrangement by osmotic pressure is also mitigated. Consequently, the performance of the sensor will have greater long-term stability.

This disclosure has been presented for purposes of illustration and description but is not intended to be exhaustive or limiting. Many modifications and variations will be apparent to those of ordinary skill in the art. The embodiments were chosen and described in order to explain principles and practical application, and to enable others of ordinary skill in the art to understand the disclosure for various embodiments with various modifications as are suited to the particular use contemplated.

Although illustrative embodiments have been described herein, it is to be understood that the embodiments are not limited to those precise embodiments, and that various other changes and modifications may be affected therein by one skilled in the art without departing from the scope or spirit of the disclosure.

What is claimed is:

1. An amperometric sensor for determination of free or combined chlorine in an aqueous sample, the sensor comprising:
a reference electrode;
an auxiliary electrode;
a housing containing an inner fill solution;
a working electrode assembly comprising:
a working electrode comprising an electrically conductive layer;
a non-compact or porous insulating layer; and
a gas or vapor permeable membrane;
wherein the non-compact or porous insulating layer is disposed between the electrically conductive layer and the gas or vapor permeable membrane;
wherein the electrically conductive layer is disposed on the non-compact or porous insulating layer such that the electrically conductive layer covers the surface of the insulating layer facing the inner fill solution and does not occlude the pores of the insulating layer;
wherein the pores of the electrically conductive layer and the non-compact or porous insulating layer align;
wherein the face of the non-compact or porous insulating layer opposite the electrically conductive layer is disposed on one side of the gas or vapor permeable membrane;
wherein the insulating material comprising the non-compact or porous insulating layer is impermeable to analyte; and
wherein the working electrode assembly is positioned over an opening in the house.

2. The amperometric sensor of claim 1, wherein the electrically conductive layer and the non-compact or porous insulating layer is formed in a pattern selected from the group consisting of a circular pattern, an angular pattern, and a porous sheet.

3. The amperometric sensor of claim 1, further comprising a support structure disposed with the housing and contacting an internal surface of the membrane.

4. The amperometric sensor of claim 1, further comprising at least one microelectrode structure.

5. The amperometric sensor of claim 1, further comprising particulates to facilitate cleaning of the working electrode of the amperometric sensor.

6. The amperometric sensor of claim 1, wherein the housing houses liters of inner fill solution.

7. The amperometric sensor of claim 1, wherein the electrode is formed as a porous sheet in intimate contact with the membrane.

8. The amperometric sensor of claim 7, wherein the porous sheet covers an entire inner surface of the membrane.

9. The amperometric sensor of claim 1, wherein the amperometric sensor comprises a plurality of electrodes bathed in the inner fill solution.

10. The amperometric sensor of claim 9, wherein the plurality of electrodes comprise a porous sheet and a patterned electrode.

11. An amperometric sensor for determination of free or combined chlorine in an aqueous sample, the sensor comprising:
a reference electrode;
an auxiliary electrode;
a housing containing an inner fill solution;
a working electrode assembly comprising:
a working electrode bathed in the inner fill solution and comprising an electrically conductive layer;
probe electronics coupled to the working electrode;
a non-compact or porous insulating layer; and
a gas or vapor permeable membrane;
wherein the non-compact or porous insulating layer is disposed between the electrically conductive layer and the gas or vapor permeable membrane;
wherein the electrically conductive layer is disposed on the non-compact or porous insulating layer such that the electrically conductive layer covers the surface of the insulating layer facing the inner fill solution and does not occlude the pores of the insulating layer;
wherein the pores of the electrically conductive layer and the non-compact or porous insulating layer align;
wherein the face of the non-compact or porous insulating layer opposite the electrically conductive layer is disposed on one side of the gas or vapor permeable membrane;
wherein the insulating material comprising the non-compact or porous insulating layer is impermeable to analyte; and
wherein the working electrode assembly is positioned over an opening in the house.

12. The amperometric sensor of claim 11, wherein the electrically conductive layer and the non-compact or porous insulating layer is formed in a pattern selected from the group consisting of a circular pattern, an angular pattern, and a porous sheet.

13. The amperometric sensor of claim 11, further comprising a support structure disposed with the housing and contacting an internal surface of the membrane.

14. The amperometric sensor of claim 11, further comprising at least one microelectrode structure.

15. The amperometric sensor of claim 11, further comprising particulates to facilitate cleaning of the working electrode of the amperometric sensor.

16. The amperometric sensor of claim 11, wherein the housing houses liters of inner fill solution.

17. The amperometric sensor of claim 11, wherein the electrode is formed as a porous sheet in intimate contact with the membrane.

18. The amperometric sensor of claim 17, wherein the porous sheet covers an entire inner surface of the membrane.

19. The amperometric sensor of claim 11, wherein the amperometric sensor comprises a plurality of electrodes bathed in the inner fill solution.

20. The amperometric sensor of claim 19, wherein the plurality of electrodes comprise a porous sheet and a patterned electrode.
